(19) United States
(12) Patent Application Publication
(10) Pub. No.: US 2007/0166394 A1
(43) Pub. Date: Jul. 19, 2007

(54) Method for manufacturing biomedical bone material with concrete characteristic

(76) Inventor: Nan-Hui Yeh, Kaohsiung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/653,217

(22) Filed: Jan. 16, 2007

(30) Foreign Application Priority Data
Jan. 17, 2006 (CN) 200610001701.2

(51) Int. Cl. A61K 35/32

(52) U.S. Cl. 424/549

(57) ABSTRACT

A method for manufacturing biomedical bone material with concrete characteristic includes mixing different sizes of biomedical bones to form bone filler with concrete feature and characteristic. The biomedical bone material thus produced is featured by a solid having particles of different sizes, and a predetermined strength.

### BACKGROUND OF THE INVENTION

1. Field of the Invention

The present invention relates to a method for manufacturing biochemical bone material, particularly for manufacturing biochemical bone material with concrete characteristic.

2. Description of the Prior Art

Calcium sulfate, generally referred to as gypsum, can be divided into anhydrous gypsum (CaSO₄), hemidrate gypsum (CaSO₄.1/2H₂O) and dihydrate gypsum (CaSO₄.2H₂O). The super hard gypsum often used in medical field is hemihydrate calcium sulfate, which can be turned into dihydrate gypsum with crystal water generated after being added with water, and be further solidified and hardened. The reaction is as following:

CaSO₄.1/2H₂O+3/2H₂O→CaSO₄.2H₂O

During the whole process, in addition to 3/2 mole water per mole of hemihydrate calcium sulfate added into the reaction, more water is needed for stirring the slurry uniformly. The more water is added, the longer time it takes for hardening and solidification. Moreover, after the reaction is completed, the water residue remained in the calcium sulfate is evaporated, forming pores in the calcium sulfate. Therefore, the more water is added, the weaker the strength of solidified calcium sulfate will be.

Calcium phosphate is a major component of human bones, and has been the common bone filler in the medical field to substitute hard bone tissues. Calcium phosphate filler is featured by its osteoconductivity, and can be surface bound to the host bone after implantation, to provide a guided bone structure. In addition, the calcium phosphate filler has a fine biocompatibility with a PH value close to that of our human body, so that it can be gradually absorbed by the main body after it is implanted into human or animal body, and bound to the host tissue and can stimulate the growth of the surrounding tissues, which therefore acts as an important bone filler. General biomedical ceramic material has an insufficient mechanical strength, especially under a complicated stress condition, and is therefore very limited in practical applications. Thus, the biomedical filler material is required to have a low loss rate, improved mechanical strength, as well as a good biocompatibility.

Bone graft application is often required in bone surgeries, because of poor healing of bone fractures, osteoma, serious trauma or osteomyelitis. However, in clinical practices, it may be difficult to get enough spongy bones for the surgery or the infection may not be suitable for immediate spongy bone grafts. Also, aging and osteoporosis problems have always been witnessed in clinical orthopaedics in recent years. As man grows older, the demand to bone substitute is increased. Partial damages resulted in diseases or caused by trauma, bone diseases can be mended on-site. But besides the traditionally used autogenous bone graft, homogenous skeleton, and processed animal skeleton, the filling material most commonly used for bone surgery is calcium sulfate-based bone cements, such as collagraft and OsteoSet bone graft substitute etc., which are greatly limited in practical applications due to factors such as material supply shortages, patient body exclusion, infection, secondary surgery, rapid dissolution, or ingrowth of soft fibrous tissues etc., and meanwhile subject to the requirements of complicated cement shapes for fitting the damages and the corresponding stresses caused thereby. Thus, the current studies are focused on how to avoid secondary surgery, reduce the loss rate of the implanted material and accelerate the growth of bone cells. It is ideal to make a filler material with a loss rate close to the growth rate of the bone, so as to avoid the ingrowth of fibrous tissues.

The bone filler material is an implantable material, either a single material or a compound of multiple materials, which can accelerate bone repair by osteogenic, osteoinductive or osteoconductive effects.

Osteogenic material contains living cells that can be differentiated into bones. Osteoconductive material helps to form a functional container frame on the surface of the bone, which can strengthen the bone formation. Osteoinductive material provides biological stimulation per se to induce the cells or transplanted cells at the implantation site to be differentiated into mature osteoblast. A material having osteogenic characteristic can be defined as having living cells that can be differentiated into bone tissues. A material having osteoconductivity attaches osseous tissue onto the surface of the material, partially as an eagle rack-like structure, which helps to bone formation. And, a material having osteoinductivity provides biologic stimulation which induces partial or transformed cells into a channel that can be differentiated into mature osteoblasts.

Thus, it is obvious that the above conventional filling material has certain defects and shortages in practical application which need to be improved.

Based on this, the present invention is proposed to reasonably and efficiently address the above problems.

### SUMMARY OF THE INVENTION

The major purpose of the present invention is to provide a method for manufacturing biomedical bone material with concrete characteristic, which includes mixing different sizes of the biomedical bone material such as hemihydrate calcium sulfates and calcium phosphates based biomedical glasses or biomedical glass-ceramics or biomedical ceramics at different proportions, to form bone filler with concrete feature and characteristic. The biomedical bone material thus produced is featured by a solid having particles of different sizes, and a predetermined strength.

To achieve the above purpose, the present invention provides a method for manufacturing a biomedical bone material with concrete characteristic, which includes: mixing a diluted acid solution with a bone cement, to form a bone cement slurry, mixing multiple fine bones into the slurry to form a bone mortar, and mixing multiple coarse bones into the mortar to generate a biomedical bone material with concrete characteristic.

The purposes, characteristics and features of the present invention will be further understood through explanation to the techniques, means and efficacies thereof with reference to the following detailed description and appended drawings, wherein the appended drawings are for reference and explanation only and should by no means deemed as to limit the present invention.

### BRIEF DESCRIPTION OF THE DRAWINGS

FIG. 1 is the flow chart of the method for manufacturing a biomedical bone material with concrete characteristic according to the present invention;

FIG. 2 is a schematic view of biomedical bones of different sizes according to the present invention;

FIG. 3 is a schematic view of mixed biomedical bones of different sizes according to the present invention; and

FIG. 4 is the scanned electron microscopic diagram of the mixed biomedical bones of different sizes according to the present invention.

### DETAILED DESCRIPTION OF PREFERRED EMBODIMENTS

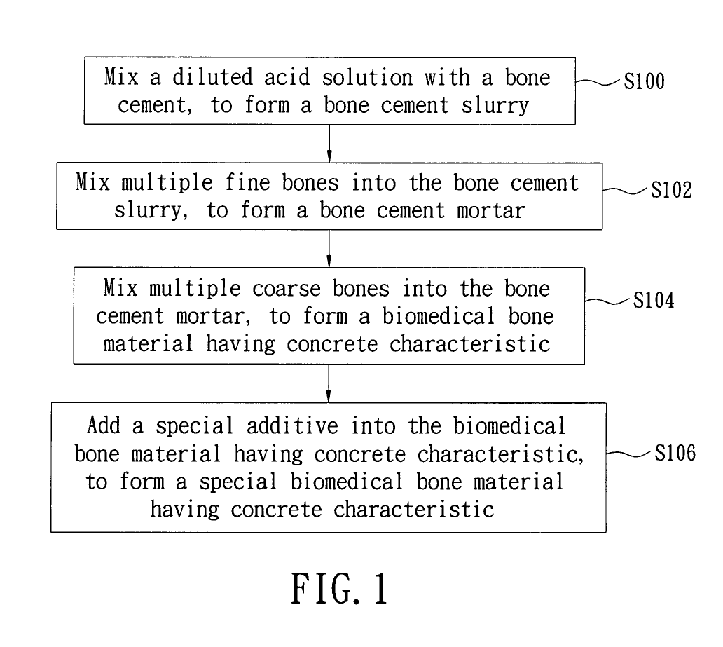

Referring to FIG. 1, a method for manufacturing a biomedical bone material with concrete characteristic is provided, which includes: mixing a diluted acid solution and a bone cement, to form a bone cement slurry (S100), mixing multiple fine bones 1 into the slurry, to form a bone cement mortar (S102); and mixing multiple coarse bones 2 into the mortar, to form a biomedical bone material with concrete characteristic (S104). It further includes a step of mixing a special additive into the biomedical bone material with concrete characteristic, to form a special biomedical bone material with concrete characteristic (S106), wherein the special additive is antibiotic or growth factor.

Said diluted acid solution is an aqueous solution of diluted phosphoric acid, and the bone cement is α,β-phase hemihydrate calcium sulfate. The fine bones 1 are dihydrate calcium sulfate particles, calcium phosphate-based biomedical glass, biomedical glass-ceramics, biomedical ceramics or PLLA. The coarse bones 2 have a size of 840˜1410 μm, wherein there are multiple medium bones 3 having a size of 590˜840 μm.

The present invention further includes: (1) smashing the reagent grade tabletted di-hydrate and hemihydrate calcium sulfate tablets and calcium phosphate-based glass or glass-ceramics or ceramics respectively with a homogenizer, and sieving them through powder shaker screens respectively through mesh 325, 200, 120 and 100 ASTM standard sieves; (2) grading the crumbs passing through the sieves and analyzing particle sizes of the fine powders by laser, and taking a proportion of powders of the minimum particle size; (3) mixing the above said two or more powders of different particles sizes to form a slurry, wherein the di-hydrate calcium sulfate is used as the substrate and the hemihydrate calcium sulfate is used as sands, both being mixed into the calcium phosphate, waiting for the mortar to be solidified.

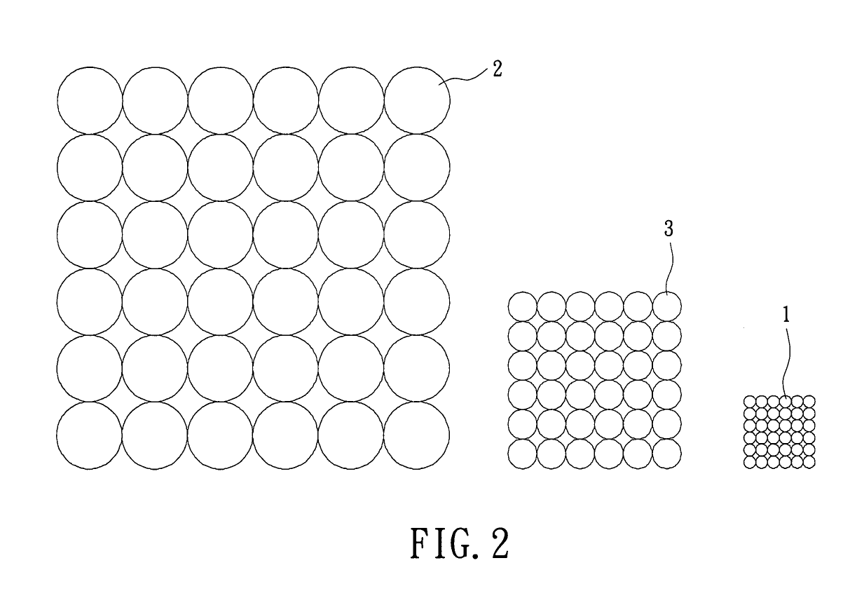

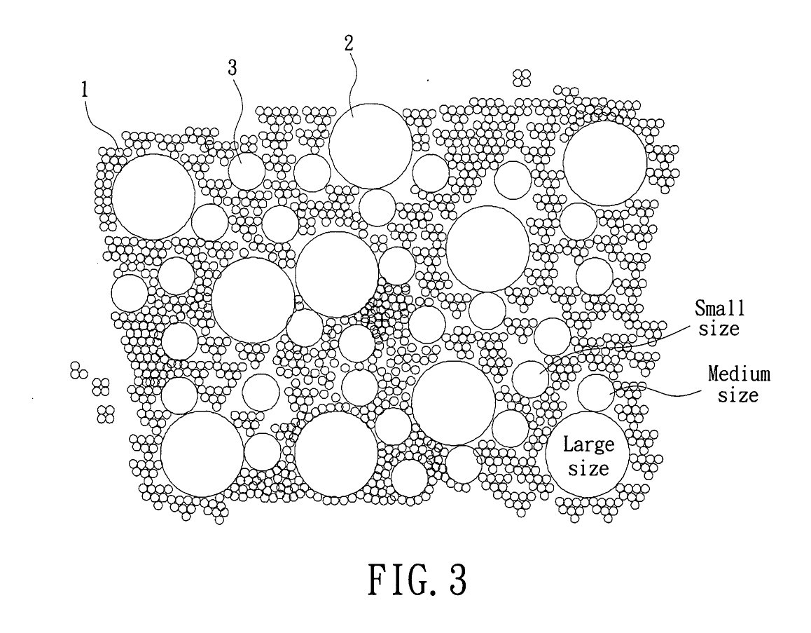

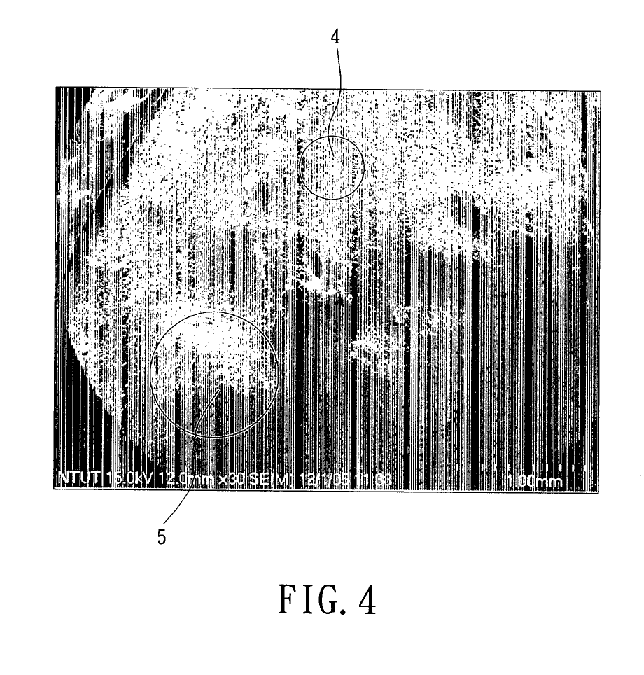

FIG. 2 and FIG. 3 have shown the mixed fine bones 1, medium bones 3 and coarse bones 2 of the present invention, and FIG. 4 shows the scanned electron microscopic diagram of the mixed biomedical bones of different particle sizes, wherein some mixtures are sands 4 and some are stones 5.

The present invention is to provide a method for manufacturing a biomedical bone material with concrete characteristic, which includes mixing different sizes of the biomedical bones, such as hemihydrate calcium sulfate, calcium phosphate-based biomedical glass or glass-ceramics or ceramics at different proportion, to form bone filler with concrete feature and characteristic. The biomedical bone material thus produced is a solid having particles of different sizes, and a predetermined strength.

However, the above disclosure is only a preferred embodiment of the present invention, and shall not be deemed as to limit the present invention. Those skilled in the arts will readily observe that numerous modifications and alterations of the present invention shall fall into the scope of the appended claims, without departing from the spirit of the present invention.

What is claimed is:

1. A method for manufacturing a biochemical bone material with concrete characteristic, including:
mixing a diluted acid solution and a bone cement, to form a bone cement slurry;
mixing multiple fine bones into the bone cement slurry, to for a bone cement mortar, and
mixing multiple coarse bones into the bone cement mortar, to form a biochemical bone material with concrete characteristic.

2. The method according to claim 1, wherein the diluted acid solution is diluted phosphoric acid.

3. The method according to claim 1, wherein the bone cement is α,β-phase hemihydrate calcium sulfate.

4. The method according to claim 1, wherein the fine bones are di-hydrate calcium sulfate particles, calcium phosphate-based biomedical glass, biomedical glass-ceramics, biomedical ceramics or PLLA.

5. The method according to claim 1, wherein the fine bones have a particle size smaller than 590 μm.

6. The method according to claim 1, wherein the coarse bones are di-hydrate calcium sulfate particles, calcium phosphate-based biomedical glass, biomedical glass-ceramics, biomedical ceramics or PLLA.

7. The method according to claim 1, wherein the coarse bones have a particle size from 840˜1410 μm.

8. The method according to claim 1, further comprising a step of mixing a special additive to the biomedical bone with concrete characteristic, to form a special biomedical bone material with concrete characteristitibiotic or growth factor.

9. The method according to claim 8, wherein the special additive is antibiotic or growth factor.
